SEQUENCE LISTING (21 sequences)

SEQ ID NO: 1 - protein, 28 aa, synthetic construct
EIAALEAKNAALKAEIAALEAKNAALKA

SEQ ID NO: 2 - protein, 28 aa, synthetic construct
KIAALKAENAALEAKIAALKAENAALEA

SEQ ID NO: 3 - protein, 33 aa, synthetic construct
SPEDEIQALEEENAQLEQENAALEEEIAQLEYG

SEQ ID NO: 4 - protein, 33 aa, synthetic construct
SPEDKIAQLKEKNAALKEKNQQLKEKIQALKYG

SEQ ID NO: 5 - protein, 33 aa, synthetic construct
SPEDEIQQLEEEIAQLEQKNAALKEKNQALKYG

SEQ ID NO: 6 - protein, 33 aa, synthetic construct
SPEDKIAQLKQKIQALKQENQQLEEENAALEYG

SEQ ID NO: 7 - protein, 33 aa, synthetic construct
SPEDEIQQLEEEISQLEQKNSQLKEKNQQLKYG

SEQ ID NO: 8 - protein, 33 aa, synthetic construct
SPEDKISQLKQKIQQLKQENQQLEEENSQLEYG

SEQ ID NO: 9 - protein, 33 aa, synthetic construct
SPEDENAALEEKIAQLKQKNAALKEEIQALEYG

SEQ ID NO: 10 - protein, 33 aa, synthetic construct
SPEDKNAALKEEIQALEEENQALEEKIAQLKYG

SEQ ID NO: 11 - protein, 33 aa, synthetic construct
SPEDEIQALEEKNAQLKQEIAALEEKNQALKYG

SEQ ID NO: 12 - protein, 33 aa, synthetic construct
SPEDKIAQLKEENQQLEQKIQALKEENAALEYG

SEQ ID NO: 13 - protein, 33 aa, synthetic construct
SPEDENQALEQKNAQLKQEIAALEQEIAQLEYG

SEQ ID NO: 14 - protein, 33 aa, synthetic construct
SPEDKNAQLKEENAALEEKIQQLKEKIQALKYG

SEQ ID NO: 15 - protein, 33 aa, synthetic construct
SPEDENQALEQEIAQLEQEIAALEQKNAQLKYG

SEQ ID NO: 16 - protein, 33 aa, synthetic construct
SPEDKNAQLKEKIAALKEKIQQLKEENQALEYG

SEQ ID NO: 17 - protein, 47 aa, synthetic construct
RFSVVKRGRKKLLYIFKQPFMRPVQTTQEEDGCSCRFPEEEEGGCEL

SEQ ID NO: 18 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 83 aa, synthetic construct
FVPVFLPAKPTTTPAPRPPTPAPTIASQPLSLRPEACRPAAGGAVHTRGLDFACDIYIWAPLAGTCGVLLLSLVITLYCNHRN

SEQ ID NO: 20 - protein, 214 aa, synthetic construct
MGCGCSSHPEDGGGSGGGSEIAALEAKNAALKAEIAALEAKNAALKAGGGSGGGSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 142 aa, synthetic construct
FVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIGGGSGGGSKIAALKAENAALEAKIAALKAENAALEAGGGSGGGS

(19) United States
(12) Patent Application Publication
(10) Pub. No.: US 2025/0222025 A1
(43) Pub. Date: Jul. 10, 2025

(54) TUNED CAR

(71) Applicant: Oslo Universitetssykehus HF, Oslo (NO)

(72) Inventors: Sébastien Wälchli, Oslo (NO); Benjamin Caulier, Oslo (NO); Christopher Forcados, Oslo (NO)

(73) Assignee: Oslo Universitetssykehus HF, Oslo (NO)

(21) Appl. No.: 18/839,855

(22) PCT Filed: Feb. 21, 2023
(86) PCT No.: PCT/EP2023/054295

§ 371 Date: Aug. 20, 2024

(30) Foreign Application Priority Data
Feb. 23, 2022 (NO) 20220243

(51) Int. Cl. A61K 35/17; A61K 40/11; A61K 40/15; A61K 40/17; A61K 40/31; A61K 40/42; A61P 35/00; C07K 14/705; C07K 14/725; C07K 16/28; C12N 15/85

(52) U.S. Cl. CPC A61K 35/17; A61K 40/11; A61K 40/15; A61K 40/17; A61K 40/31; A61K 40/4211; A61K 40/4224; A61P 35/00; C07K 14/7051; C07K 14/70578; C07K 16/2803; C07K 16/2896; C12N 15/85; A61K 2239/13; A61K 2239/17; A61K 2239/21; A61K 2239/29; C07K 2317/622

(57) ABSTRACT

Provided herein are cells expressing a first and a second chimeric protein in the cell membrane. Such cells can solve expression problems of CAR constructs caused by these CAR construct's ability to recognize unwanted internal epitopes or exhibiting unwanted signalling due to misfolding/scFv aggregation. The first chimeric protein comprises an extracellular antigen binding unit and an intracellular dimerization domain. The second chimeric protein comprises a lipid anchoring domain, an intracellular dimerization domain and a signaling domain. Accordingly, the expression of the two proteins allows them to translocate to the cell membrane without much interference and subsequently gain signaling capacity when colocalized at the cell membrane.

### TECHNICAL FIELD OF THE INVENTION

The invention is related to the field of cell therapy. In particular, it relates to cell therapy, chimeric proteins, nucleic acids encoding the chimeric proteins, and their utility for treatment of cancer.

### BACKGROUND

Chimeric antigen receptors (CARs) have been approved in cell therapeutic products. In order to achieve a therapeutic CAR cell, the cell needs to express the CAR in a sufficient amount at the cell membrane, and the antigen binding unit has to possess sufficient affinity and specificity for the target antigen.

Expression problems frequently occur, but failed experiments and attempts are rarely published. Such problems may be caused by instability of the antigen binding unit, due to misfolding, aggregation with other proteins or specific/unspecific recognition of internal epitopes in intracellular compartments. Indeed, CAR molecules are, as most of transmembrane receptors, translocated through the cellular membrane at the endoplasmic reticulum (ER) level, where they might be assisted in their folding by chaperones. It is not well determined how CAR molecules are trafficked to the cell membrane, but it is tempting to speculate that they follow the route of any receptor guided by sugar modifications to reach the Golgi apparatus and are finally sent to the cell membrane.

Some CAR molecules have been reported to cause tonic signaling, which is attributed to their capacity to signal without encountering their cognate target. It is not well determined where in the cell this tonic signaling occurs.

CAR tonic signaling may be defined as a non-coordinated and sustained activation of the cell in either a ligand-independent or dependent fashion. In the absence of spatial and/or temporal control of CAR cell surface expression, this constitutive or chronic cell signalling may have a substantial deleterious impact on CAR cell effector function.

Recent evidence have shown that the binding part of the CAR molecule, usually a single chain variable fragment (scFv), could in some instance aggregate with other molecules, which would lead to a non-specific signaling. Landoni et al. Cancer Immunol Res (2021) 9 (4): 441-453 demonstrated that tonic signaling of a CAR molecule was due to self-aggregation of an unstable scFv and that it could be abrogated by stabilization of the framework regions. It has been further shown that guided stabilization of the molecule by amino acid exchange could repress this effect. Thus, incorrect folding of the CAR molecule may be one underestimated cause for the tonic signaling.

Furthermore, CAR cells will likely need to sustain their activity over time in order to provide a therapeutic effect in vivo. However, if the CARs are constitutively activated, the cells may be exhausted before a therapeutic effect can be achieved. It is therefore not trivial, but very desirable to obtain novel CAR expressing cells able to provide a therapeutic effect in vivo.

### SUMMARY OF THE INVENTION

Provided herein are cells expressing a first and a second chimeric protein in the cell membrane. These cells can solve expression problems of CAR constructs caused by the CARs recognizing internal epitopes or caused by CAR stimulation of cells due to self-aggregation in the intracellular organelles of the cells such as the ER. Furthermore, they can reduce unwanted tonic CAR signaling and thereby avoiding exhaustion of the cells. This may in turn improve in vivo persistence and/or therapeutic efficacy.

The first chimeric protein comprises an extracellular antigen binding unit and an intracellular dimerization domain. The second chimeric protein comprises a lipid anchoring domain, an intracellular dimerization domain and a signaling domain wherein the two chimeric proteins dimerize at the cell membrane.

The first chimeric protein will be synthesized as a transmembrane protein in the ER and sent to the cell membrane without being able to signal due to lack of a signaling domain on the cytosolic part. The second chimeric protein will be a signalling domain fused to plasma encored domain, and will therefore be synthesized in the cytosol and transported to the cell membrane through a mechanism independent of the ER machinery. Thus, the expression of the two chimeric proteins will take place at different locations within the cell which will counteract potential CAR mediated tonic signal from the ER or the other intracellular organelles. The two proteins will only meet at the plasma membrane were they will be form a complete CAR protein, able to signal.

Therefore, the present technical solution reduces or prevents unwanted signaling as long as the two chimeric proteins are separated and enable activation signaling to take place once the two chimeric proteins dimerize at the cell membrane.

We thus provide in a first aspect a cell expressing a first and a second chimeric protein in the cell membrane;
wherein the first chimeric protein comprises, from N-terminal to C-terminal, an antigen binding unit, a transmembrane domain and a first dimerization domain, but no functional CD3ζ signaling domain and
wherein the second chimeric protein comprises, from N-terminal to C-terminal, a lipid-anchoring domain, a second dimerization domain and a signaling domain.

In one embodiment according to the first aspect
wherein the first chimeric protein consists of, from N-terminal to C-terminal, an antigen binding unit, a transmembrane domain and a first dimerization domain and
wherein the second chimeric protein comprises, from N-terminal to C-terminal, a lipid-anchoring domain, a second dimerization domain and a signaling domain.

In one embodiment according to the first aspect
wherein the first chimeric protein consists of, from N-terminal to C-terminal, an antigen binding unit, a transmembrane domain and a first dimerization domain and,
wherein the second chimeric protein consists of, from N-terminal to C-terminal, a lipid-anchoring domain, a second dimerization domain and a signaling domain.

In one embodiment according to the first aspect the antigen binding unit is a scFv.

In one embodiment according to the first aspect the cell is a T cell, an NK cell or a Macrophage.

In one embodiment according to the first aspect the signaling domain in the second chimeric protein comprises a CD3ζ signaling domain.

In one embodiment according to the first aspect the signaling domain in the second chimeric protein comprises a costimulatory domain and a CD3ζ signaling domain.

In one embodiment according to the first aspect the signaling domain in the second chimeric protein comprises a 4-1BB costimulatory domain and a CD3ζ signaling domain.

In one embodiment according to the first aspect the first chimeric protein comprises a hinge domain between the antigen binding unit and the transmembrane domain.

In one embodiment according to the first aspect the first dimerization domain may specifically bind to the second dimerization domain with sufficient affinity to convey a signal upon binding of target epitope.

In one embodiment according to the first aspect the first dimerization domain has a net positive charge and the second dimerization domain has a net negative charge.

In one embodiment according to the first aspect the first dimerization domain has a net negative charge and the second dimerization domain has a net positive charge.

In one embodiment according to the first aspect the first dimerization domain is represented by SEQ ID NO 2, 4, 6, 8, 10, 12, 14 or 16.

In one embodiment according to the first aspect the second dimerization domain is represented by SEQ ID NO 1, 3, 5, 7, 9, 11, 13 or 15.

In one embodiment according to the first aspect the first chimeric protein comprises a costimulatory domain.

In one embodiment according to the first aspect the second chimeric protein comprises two costimulatory domains.

In one embodiment according to the first aspect the antigen binding unit has specific and/or unspecific affinity for internal epitopes in the intracellular compartments which would cause expression problems of conventional CAR constructs.

In one embodiment according to the first aspect wherein expression of the antigen binding unit comprised in a conventional CAR construct is toxic to the cell.

In a second aspect there is provide a nucleic acid encoding the first and/or second chimeric protein as defined in the first aspect and any embodiments thereof.

In a third aspect there is provided a pharmaceutical composition comprising the cells as defined in the first aspect and any embodiments thereof, or the nucleic acid according to the second aspect.

In a fourth aspect the invention provides a method of treating cancer comprising the step of administering the pharmaceutical composition according to the third aspect to a patient in need thereof, and wherein the antigen binding unit may specifically bind to a surface antigen on cancer cells under physiological conditions.

In a fifth aspect the invention provides a method for bypassing expression problems of CARs by transducing a cell with the nucleic acid as defined in the second aspect.

### BRIEF DESCRIPTION OF THE DRAWINGS

FIG. 1. The Tuned CAR-system is active and specific. A T cell line J76 stably transduced with a NFTA (Nuclear factor of activated T-cells) eGFP Reporter expressing a GFP protein downstream of a NFAT response element (J76 NFAT^{GFP} reporter) was either transduced with the depicted CAR constructs shown in figure A and B or left untransduced. Cells cocultured with (A) BL-41 (CD19+ and CD37^{high}) or (B) THP-1 (CD37^{low}) target cells show specific activation. Tuned CD19CAR is efficient in activating T cell. Tuned CD37CAR is as efficient as CD37CAR in activating T cell.

FIG. 2. The Tuned CAR-system reduces tonic signaling. T cell line J76 stably transduced to express GFP protein downstream of a NFAT response element (J76 NFAT^{GFP} reporter) was either transduced with the depicted CAR constructs or left untransduced. (A) Viability was compared at day 11 and shows reduced toxicity using a Tuned CD37CAR construct compared to CD37CAR construct. (B) Therefore, Tuned CD37CAR cells expands better. (C) It is noted that the Tuned CD37CAR construct is expressed to the same extent as the CD37CAR construct. (D) Importantly, The Tuned CD37CAR construct triggers less basal T cell activation than a CD37CAR construct which may explain the advantageous features. The results are based on 2 independent experiments (n=2).

FIG. 3. The Tuned CAR-system is active in primary T cells. Two donors' PBMCs (n=2) were either transduced with the depicted CAR constructs or left untransduced and expanded for 10 days. Expanded CAR-T cells cocultured with BL-41 (CD19+) target cells show antigen-specific exposure of CD107 marker at the membrane reflecting cytotoxic degranulation. Cells transduced with the Tuned CD19CAR construct is efficient in activating the T cell upon antigen recognition and further dampens unspecific activation in absent of its target (BL-41 CD19^{KO} (knock-out)) compared to a CD19CAR transduced cell.

FIG. 4. Tuned CAR-system is resistant to multiple target challenges. Two donors' PBMCs (n=2) were either transduced with depicted CARs or left untransduced and expanded for 10 days. (A) Expanded CAR-T cells cocultured with BL-41 (CD19+ CD37+) target cells show antigen-specific killing over increasing incubation time. Tuned CD37CAR is as efficient as CD37CAR in killing assay. (B) CAR T cells were then rechallenged weekly with irradiated CD19+ CD37+ lymphoma cell lines over 5 weeks to assess the sustainability of killing efficiency (exhaustion of T cells). Tuned CD37CAR killing performance is stronger and lasts longer than CD37CAR or CD19CAR. Statistical test (one-way ANOVA) demonstrated a difference between Tuned CD37CAR with CD37CAR at week 5.

FIG. 5. Tuned CD37CAR maintains activity and specificity. Tuned CD37CAR is as efficient as CD37CAR in killing of CD37+BL-41 (Burkitt Lymphoma) and THP-1 (AML) cell line. Two donors' PBMCs (n=2) were either transduced with the depicted CAR molecules or left untransduced and expanded for 10 days. Expanded CAR-T cells cocultured with CD37+ target cells show antigen-specific killing over increasing effectors to target (E:T) ratios. Tuned CD37CAR is as efficient as CD37CAR in killing assay and shows no unspecific killing/tonic activity when target is knocked out (THP-1 CD37KO) by CRISPR-Cas9.

FIG. 6. Tuned CD37CAR improves expansion. Two donors' PBMCs (n=2) were either transduced with depicted CAR molecules or were mock transduced and expanded for 12 days. Expanding CAR-T cells were counted every 4/5 days. Tuned CD37CAR shows better expansion than CD37CAR known to be difficult to expand and may thus serve to circumvent this kind of hurdle.

FIG. 7. Tuned CAR counteracts CAR Toxicity. Anti-GRP94 CAR molecules are toxic to a cell, probably due to recognition of the endogenous GRP94 protein at the ER, therefore expression of these CARs lead to cell death. In contrast to T cells transduced with conventional second generation GRP94 CAR, T cells transduced with the Tuned CAR-system are able to express CARs targeting GRP94 and remain viable.

FIG. 8A, 8B. The Tuned CAR-system is metabolically less tonic at steady state. Healthy donor's PBMCs were transduced or not (mock) with second generation CD19 CAR or a Tuned CD19 CAR and expanded for 17 days. T cells were then evaluated for their metabolic activity using a Seahorse XF instrument which measures Oxygen Consumption Rate (OCR) and Extra Cellular Acidification Rate (ECAR) at steady state. Tuned CD19CAR showed reduced OCR/ECAR reflecting a metabolically less active state than CD19CAR. Thus, the Tuned CAR-system likely dampens known CD19CAR tonic signaling.

FIG. 9a, 9b, 9c, 9d, 9e, 9f, 9G, 9H and 9I visualizes several examples of the Tuned CAR-system, i.e. a cell expressing the first and the second chimeric protein in the cell membrane.

FIG. 10 visualizes how the first chimeric protein is synthesized in cytosol and the second chimeric protein is synthesized in ER.

FIG. 11 visualizes how a Tuned CAR-system (i.e. a cell expressing the first and the second chimeric protein in the cell membrane) are less toxic than conventional second generation CARs in primary T cells.

The cells were either non-transduced (control, red), transduced with a CD19CAR (orange) or GRP94CAR (green). Upper panel shows expression of the Tuned CAR-system and the corresponding viability after 9 days of culture. The expression is well sustained, and cells grow. Lower panel shows Regular CARs expression and the corresponding viability after 48 h. The expression is low and decreasing overtime, all cells are usually dead after 5 days.

FIG. 12 T cells according to the present disclosure were able to kill a GRP94+ target, the model breast cancer cell line MCF-7. As shown, the Tuned CAR-system was effective and potent compared to the controls (FIG. 12, green versus blue/red).

### DETAILED DESCRIPTION

Without being bound by theory, the first and the second chimeric protein can be expressed in cells without interfering with each other during their synthesis.

A Tuned CAR-system means a cell expressing a first and a second chimeric protein, as defined herein, in the cell membrane. Accordingly, a Tuned GRP94CAR means a Tuned CAR-system with specific affinity for cells expressing GRP94 at the cell surface. Accordingly, a Tuned CD37CAR means a Tuned CAR-system with specific affinity for cells expressing CD37 at the cell surface. Accordingly, a Tuned CD19CAR means a Tuned CAR-system with specific affinity for cells expressing CD19 at the cell surface.

The Tuned CAR-system described herein is a technical solution that is particularly suitable for use when the conventional CAR construct is toxic to the cell, i.e. expression of the CAR construct kills the cell thereby decreasing its therapeutic potential.

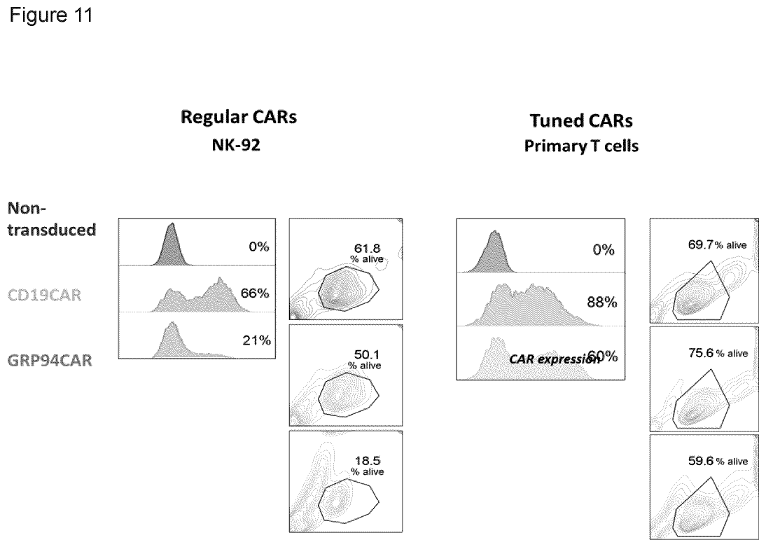

An example of a conventional CAR construct that is, when expressed, is toxic to the cell, is a conventional CAR construct targeting GRP94. The inventors have shown that cells expressing Tuned CAR-system targeting GRP94 increases cell viability compared to expression of a conventional GRP94CAR, cf. FIG. 11.

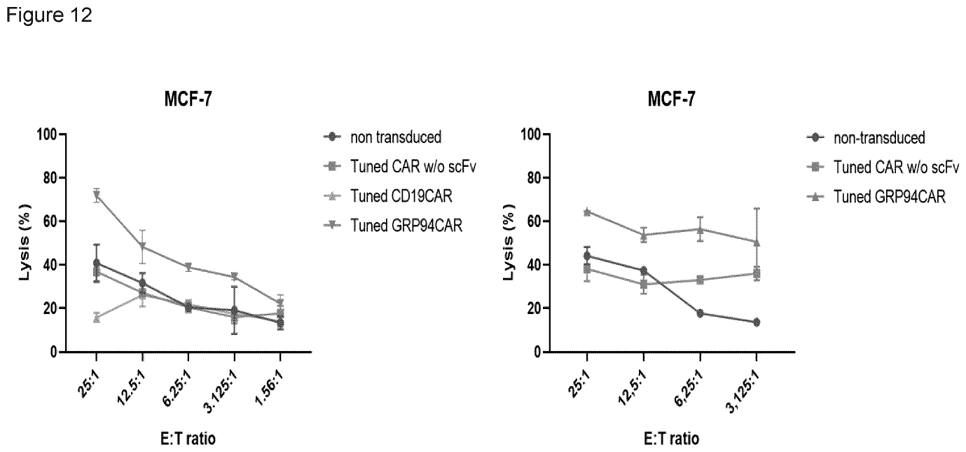

Further the present disclosure demonstrates that T cells expressing Tuned GRP94CAR were able to kill a GRP94+ target, the model breast cancer cell line MCF-7, cf. FIG. 12.

### The First Chimeric Protein

The first chimeric protein comprises, from N-terminal to C-terminal, an antigen binding unit, a transmembrane domain and a dimerization domain, but no functional CD3ζ signaling domain. Due to the signal peptide at the N-terminal, transmembrane and the juxtamembrane domains, such proteins will be synthesized and translocated to the cell membrane through the endoplasmic reticulum and trafficked to the plasma membrane through the Golgi apparatus.

As used herein, an “antigen binding unit” is a protein moiety able to bind an extracellular target epitope under physiological conditions. The antigen binding unit herein may be able to bind an extracellular target epitope under physiological conditions in a tumor environment.

The antigen binding unit may comprise an antibody light chain variable domain (VL) and an antibody heavy chain variable domain (VH). Such variable domains are well-known for skilled persons. An antigen binding unit comprising a VL and a VH are often called a single chain Fragment variable, scFv. Notably, single-domain antibodies isolated from camelids are also known and useful sources for antigen binding units, they are called nanobodies. In general, the antigen binding unit may also be any protein or peptide moiety able to specifically bind to a cellular surface target under physiological conditions. Such binding units may be a receptor domain or its cognate ligand, for example PD1-receptor and its ligand PD-L1, NKG2D-receptor and its ligand MICA, Interleukin-13 receptor alpha-2 and its ligand IL-13 etc

Each VL and VH herein comprises three complementarity determining regions (CDRs) flanked by framework sequences. The framework sequences may be human, humanized or murine sequences.

A Framework1 sequence is N-terminal to the CDR1, a Framework2 sequence is located between CDR1 and CDR2, while a Framework3 sequence is located between CDR2 and CDR3.

Accordingly, both a VL and VH can be roughly visualized as follows, with the CDRs boxed and the N-terminal indicated as N-:

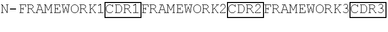

FRAMEWORK4

The VH and VL may be connected by a disulphide bridge or a peptide linker. Alternatively, the two chains may be embedded in a Fab-fragment of an antibody or an antibody as such. In one embodiment, the antigen binding unit comprises or consists of VL-linker-VH. In another embodiment, the antigen binding unit comprises or consists of VH-linker-VL. Such antigen binding units are often referred to as single chain Fv-fragments (scFv's).

The linker in scFv's may have a certain length in order to allow the VH and VL to form a functional antigen binding unit. In one embodiment, the linker comprises 10 to 30 amino acid residues. In one embodiment, the linker comprises 15 to 25 glycine and/or serine residues.

In one embodiment, each VL and VH herein comprises three CDRs flanked by human framework sequences. Human framework sequences are structurally conserved regions that normally tend to form a β-sheet structure delicately positioning the CDRs for specific binding to the target antigen under physiological conditions. Many human framework sequences are available from known human antibodies and from the international ImMunoGeneTics information system (IMGT) online database (see Giudicelli et al, Nucleic Acids Research, 2006, Vol. 34, Database issue D781-D784), but the term also covers human framework sequences comprising amino acid substitutions.

In one embodiment, the human framework sequences are mature human framework sequences available from known human antibodies. Without being bound by theory, such framework sequences may convey very low risk of triggering unwanted immunogenic responses against the antigen binding unit, and at the same time increase the likelihood of obtaining stable binding units which are well expressed in cellular systems.

The antigen binding unit may be directly attached to a transmembrane domain. However, the first chimeric protein may comprise a linker domain connecting the antigen binding unit to the transmembrane domain. Said linker may be a hinge domain. The hinge domain may thus affect the sterical conformation of the antigen binding unit. This may in turn affect the ability of the first chimeric protein to bind the target epitope and subsequently trigger signaling into an immune cell. If the target epitope is located too far from the cell membrane of the target cell or if the target epitope is otherwise hidden, the immune cell expressing the first and second chimeric protein may not be efficient. Accordingly, it is preferred that the target epitope is sufficiently accessible for immune cells expressing the first and second chimeric protein.

The transmembrane domain connects the extracellular domains to an intracellular domain. Both the antigen binding unit and hinge domain are extracellular domains, i.e. that they generally face the extracellular environment when expressed in the cell membrane of an immune cell. As used herein, “transmembrane domain”, means the part of the first chimeric protein which tends to be embedded in the cell membrane when expressed by an immune effector cell. Suitable transmembrane domains are well known for skilled persons. In particular, transmembrane domains from the human proteins CD8α, CD28 or ICOS may be used. The transmembrane domain is believed to convey a signal into immune cells upon binding of a target by the antigen binding unit.

The transmembrane domain is connected to a dimerization domain, either directly or via a linker. The dimerization domain in the first chimeric protein can be any protein moiety providing sufficient specific affinity for the dimerization domain in the second chimeric protein. Such dimerization domains are known for skilled persons, and they may facilitate the co-localization of first and second chimeric protein. The dimerization domains may thus be coiled-coil, knob-in-hole constructs, leucine zippers, ligand-receptor pairs etc. As described by Lebar et al in Nature Chemical Biology volume 16, pages 513-519 (2020), coiled-coil (CC) dimers are among the smallest protein dimerization domains, in which polypeptide helices interact along the complementary surface. CC homodimers such as variants of the dimerization domain of a yeast transcription factor GCN4 and leucine zippers have already been used to guide protein interactions. However, heterodimerization domains may be more precise and efficient in guiding interactions between different protein partners. The most frequently used CC heterodimeric peptide pair is based on peptide partners that are positively (basic or K peptide) and negatively charged (acidic or E peptide). Eight such pairs are disclosed herein: SEQ ID NO: 1 and 2, SEQ ID NO: 3 and 4, SEQ ID NO: 5 and 6, SEQ ID NO: 7 and 8, SEQ ID NO: 9 and 10, SEQ ID NO: 11 and 12, SEQ ID NO: 13 and 14 and SEQ ID NO: 15 and 16.

Each of the first and second chimeric protein may comprise one or more of such dimerization domains.

Notably, the first chimeric protein in the present disclosure does not comprise a functional CD3ζ signaling domain, i.e. a CD3ζ signaling domain comprising the three immunoreceptor tyrosine-based activation motifs (ITAMs) intact.

Accordingly, in one preferred embodiment, the first chimeric protein comprises no functional ITAM.

In another embodiment, the first chimeric protein comprises no more than one functional ITAM.

In another embodiment, the first chimeric protein comprises no more than two functional ITAMs.

### The Second Chimeric Protein

The second chimeric protein comprises, from N-terminal to C-terminal, a lipid-anchoring domain, a dimerization domain and a signaling domain. Such proteins will be synthesized in the cytosol.

The lipid-anchoring domain is a protein moiety that will be covalently attached to a lipid. The main types of lipid-anchored proteins include prenylated proteins, fatty acylated proteins and glycosylphosphatidylinositol-linked proteins (GPI). The lipid anchoring domain specifically target the second chimeric protein to the inner leaflet of the plasma membrane.

Fatty acylated proteins are proteins that have been post-translationally modified to include the covalent attachment of fatty acids at certain amino acid residues. The most common fatty acids that are covalently attached to the protein are the saturated myristic acid and palmitic acid. N-myristoylation (i.e. attachment of myristic acid) is generally an irreversible protein modification that typically occurs during protein synthesis in which the myristic acid is attached to the α-amino group of an N-terminal glycine residue through an amide linkage. S-palmitoylation (i.e. attachment of palmitic acid) is a reversible protein modification in which a palmitic acid is attached to a specific cysteine residue via thioester linkage.

The lipid-anchoring domain in the second chimeric protein is connected to a dimerization domain, either directly or indirectly via a linker. The length of the linker can be chosen for facilitating the positioning of the first dimerization domain to interact with a second dimerization domain in the second chimeric protein.

The first and second dimerization domain may be identical, but they may also be different. The first and second dimerization domain may have opposite net charge and/or contain cysteine residues suitable for forming a cysteine bridge. Linkers flanking the dimerization domains may be used for positioning the dimerization domains at locations where they can interact with each other.

The “intracellular domain” refers to a part of the first and/or second chimeric protein located inside the immune cell. These domains may participate in conveying the signal upon binding of the target. A variety of signaling domains are known, and they can be combined and tailored to fit the endogenous signaling machinery in the immune cells.

In one embodiment the intracellular signaling domain of the second chimeric protein comprises a “signal 1” domain like the signaling domains obtainable from the human proteins CD3ζ, FcR-γ, CD3ε etc. In general, it is believed that “signal 1”domains (e.g. the CD3ζ signaling domain) convey a signal upon antigen binding of conventional CARs.

In another embodiment, the intracellular signaling domain comprises a co-stimulatory domain. Such domains are well known and often referred to as “signal 2” domains, and they are believed to, subsequently of “signal 1” domains, convey a signal via costimulatory molecules. The “signal 2” is important for the maintenance of the signal and the survival of the cells. Examples of such commonly used human “signal 2” domains include 4-1BB costimulatory signaling domain, CD28 signaling domain and ICOS signaling domain.

The second chimeric protein may comprise one or two co-stimulatory domains and they may comprise a CD3ζ-signaling domain. The first chimeric protein may comprise one or two co-stimulatory domains.

### Cells

The suitable cells for use according to the present disclosure are immune cells known to be successfully transduced with nucleic acids encoding CARs. Such cells include T-cells, Natural Killer (NK)-cells, and Macrophages. Human cells are preferred when human therapies are intended, and the source may be autologous or allogenic.

The immune cells expressing the first and second chimeric protein herein may be isolated from a patient or a compatible donor by leukapheresis or other suitable methods. Such primary cells may for example be T cells or NK cells. In particular, autologous T cells (both cytotoxic T cells, T helper cells or mixtures of these) may be transduced with nucleic acids encoding the first and second chimeric protein before a pharmaceutical composition comprising the cells is administered back to the patient. The immune cells expressing the first and second chimeric protein may also be cell lines suitable for clinical use like NK-92 cells. Of course, the preferred cells are human when the intended patient is human.

Clearly, the cells provided herein may express more than one type of the first and/or second chimeric protein. For example, the cells may express two or more types of the first chimeric protein, each with its own antigen binding unit. This may allow multiple targeting, i.e. providing a cell wherein optimal activity requires the presence of multiple antigens on the target cell. The technical effect of such cells would be increased specificity. In other examples, the cells may express two or more types of the first chimeric protein, each with the same antigen binding unit.

The cells provided herein may express more than one type of the second chimeric protein. For example, the cells may express two or more types of the second chimeric protein, each type may have their own signaling domain. This may allow a stronger activation of the immune cells, i.e. providing a cell wherein there is an increased cytotoxicity and/or cytokine production ability. The technical effect of such cells would be increased clearance of the target cells.

Pharmaceutical Compositions and their Administration

The pharmaceutical compositions herein can be a composition suitable for administration of therapeutic cells to a patient. The most common administration route for therapeutic cells is intravenous administration. Accordingly, said pharmaceutical compositions may for example be sterile aqueous solutions with a neutral pH. For example, a patient's peripheral blood mononuclear cells may be obtained via a standard leukapheresis procedure. The mononuclear cells may be enriched for T cells, before transducing them with a retroviral/lentiviral vector or mRNA encoding the first and second chimeric protein. Said cells may then be activated with anti-CD3/CD28 antibody coated beads. The transduced T cells may be expanded in cell culture, washed, and formulated into a sterile suspension, which can be cryopreserved. If so, the product is thawed prior to administration.

In situations where the tumor is localized, different administrations methods may be used to improve efficacy. For example, regional or local administration rather than systemic administration of therapeutic cells might enhance efficacy.

The pharmaceutical compositions may comprise a pharmaceutically effective dose of the immune cells herein. A pharmaceutically effective dose may for example be in the range of 1×10⁶ to 1×10¹⁰ immune cells. A pharmaceutically effective dose may for example be in the range of 1×10⁷ to 1×10⁹ T cells per Kg expressing the first and second chimeric protein. A pharmaceutically effective dose may for example be in the range of 1×10⁷ to 1×10⁹ NK cells per Kg expressing the first and second chimeric protein.

For efficient expression of the first chimeric protein in immune cells, a conventional leader peptide may be introduced N-terminally for facilitating location in the cell membrane. The leader peptide is believed to be trimmed off and will likely not be present in the functional first chimeric protein in the cell membrane. Such leader peptides are well known for skilled persons.

### Nucleic Acids

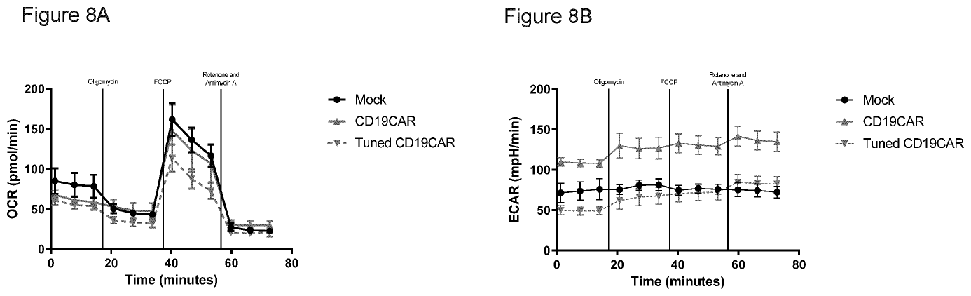

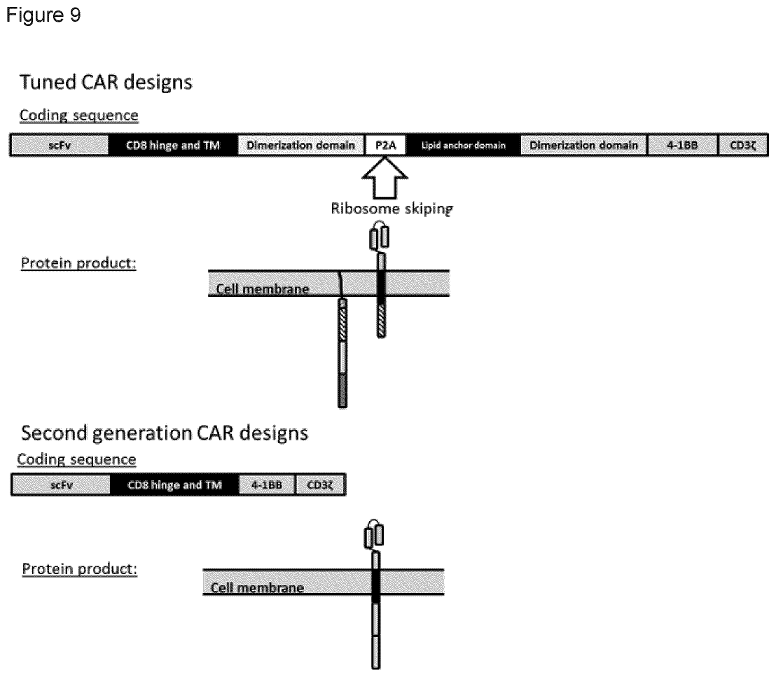

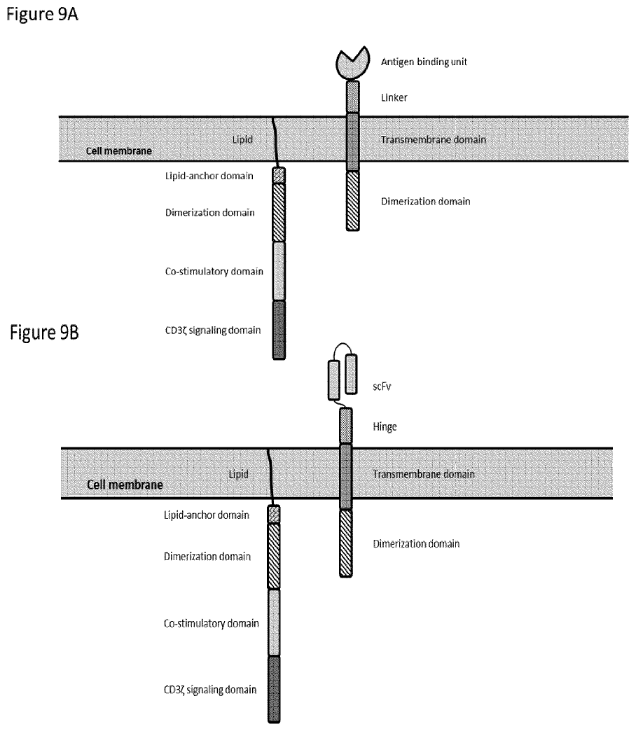

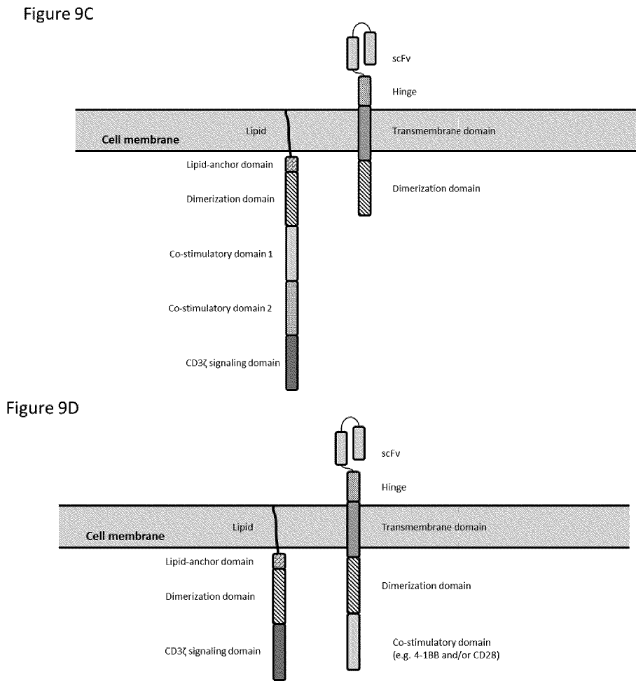

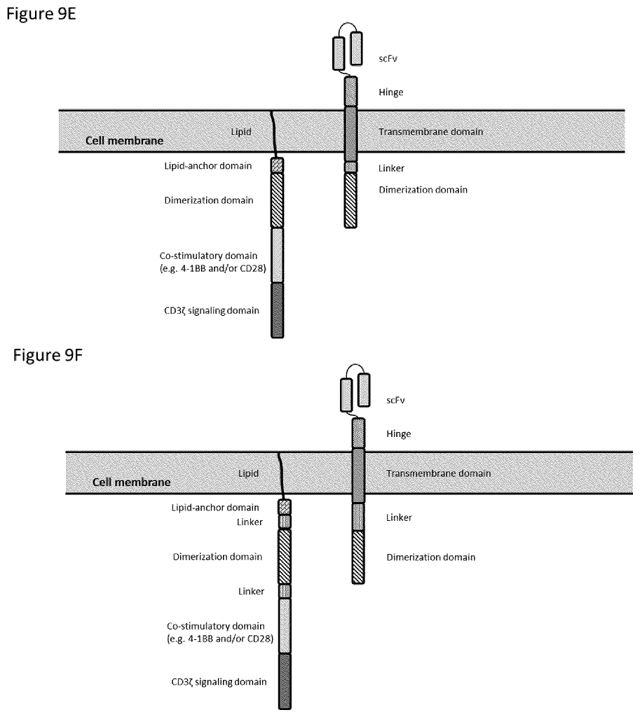

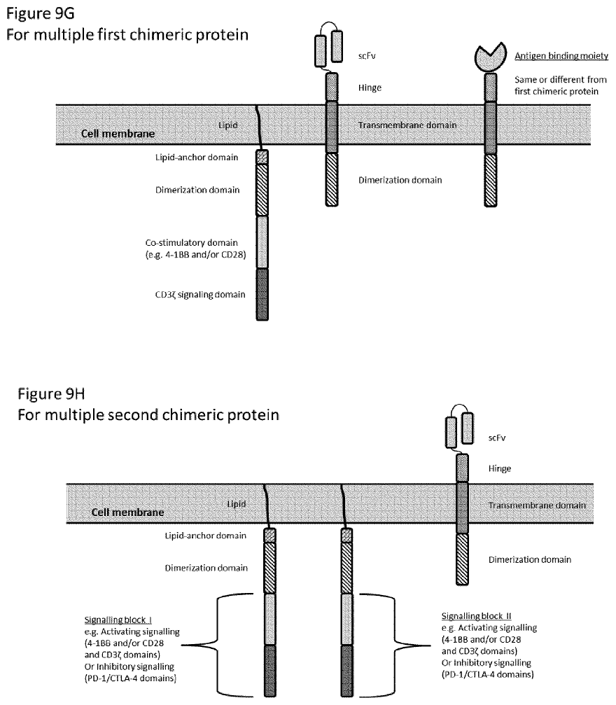

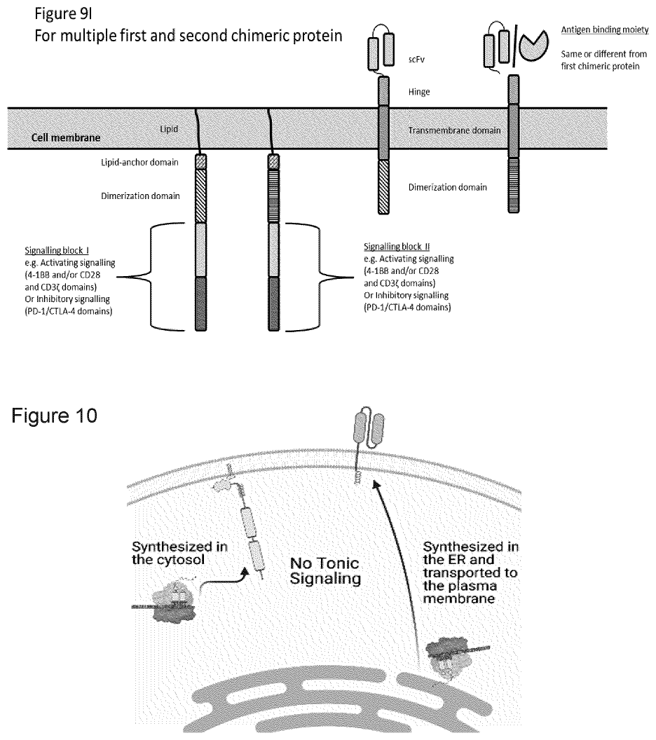

The nucleic acids encoding the first and second chimeric protein can be in the form of well-known RNA e.g. mRNA, or DNA expression vectors. For stoichiometric expression of the first and second chimeric protein, polycistronic expression vectors may be used. The vectors may encode a well-known ribosomal skipping sequence between the first and the second chimeric protein, e.g. a 2A peptide, for illustration see FIG. 8B, “P2A”. Surrogate markers such as (fluorescent proteins) or truncated (i.e CD34, NGFR, CD19) or not synthetic transmembrane protein may be used to track cells.

### SEQUENCES

SEQ ID NO: 1 and 2, SEQ ID NO: 3 and 4, SEQ ID NO: 5 and 6, SEQ ID NO: 7 and 8, SEQ ID NO: 9 and 10, SEQ ID NO: 11 and 12, SEQ ID NO: 13 and 14 and SEQ ID NO: 15 and 16: represents 8 coiled-coil heterodimeric peptide pairs.

SEQ ID NO: 1
EIAALEAKNAALKAEIAALEAKNAALKA
SEQ ID NO: 2
KIAALKAENAALEAKIAALKAENAALEA
SEQ ID NO: 3
SPEDEIQALEEENAQLEQENAALEEEIAQLEYG
SEQ ID NO: 4
SPEDKIAQLKEKNAALKEKNQQLKEKIQALKYG
SEQ ID NO: 5
SPEDEIQQLEEEIAQLEQKNAALKEKNQALKYG
SEQ ID NO: 6
SPEDKIAQLKQKIQALKQENQQLEEENAALEYG
SEQ ID NO: 7
SPEDEIQQLEEEISQLEQKNSQLKEKNQQLKYG
SEQ ID NO: 8
SPEDKISQLKQKIQQLKQENQQLEEENSQLEYG
SEQ ID NO: 9
SPEDENAALEEKIAQLKQKNAALKEEIQALEYG
SEQ ID NO: 10
SPEDKNAALKEEIQALEEENQALEEKIAQLKYG
SEQ ID NO: 11
SPEDEIQALEEKNAQLKQEIAALEEKNQALKYG
SEQ ID NO: 12
SPEDKIAQLKEENQQLEQKIQALKEENAALEYG
SEQ ID NO: 13
SPEDENQALEQKNAQLKQEIAALEQEIAQLEYG
SEQ ID NO: 14
SPEDKNAQLKEENAALEEKIQQLKEKIQALKYG
SEQ ID NO: 15
SPEDENQALEQEIAQLEQEIAALEQKNAQLKYG
SEQ ID NO: 16
SPEDKNAQLKEKIAALKEKIQQLKEENQALEYG
SEQ ID NO 17: 4-1BB:
RFSVVKRGRKKLLYIFKQPFMRPVQTTQEEDGCSCRFPEEEEGGCEL
SEQ ID NO 18: CD3ζ:
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR
SEQ ID NO 19: CD8α-hinge-TM:
FVPVFLPAKPTTTPAPRPPTPAPTIASQPLSLRPEACRPAAGGAVHTRG
LDFACDIYIWAPLAGTCGVLLLSLVITLYCNHRN
SEQ ID NO 20: second chimeric protein
MGCGCSSHPEDGGGSGGGSEIAALEAKNAALKAEIAALEAKNAALKAGG
GSGGGSRFSVVKRGRKKLLYIFKQPFMRPVQTTQEEDGCSCRFPEEEEG
GCELRVKFSRSADAPAYQQGQNQLYNELNLGRREEYDVLDKRRGRDPEM
GGKPRRKNPQEGLYNELQKDKMAEAYSEIGMKGERRRGKGHDGLYQGLS
TATKDTYDALHMQALPPR
SEQ ID NO 21: first chimeric protein
SCFV-
FVPVFLPAKPTTTPAPRPPTPAPTIASQPLSLRPEACRPAAGGAVHTRG
LDFACDIYIWAPLAGTCGVLLLSLVITLYCNHRNRFSVVKRGRKKLLYI
GGGSGGGSKIAALKAENAALEAKIAALKAENAALEAGGGSGGGS

### Examples

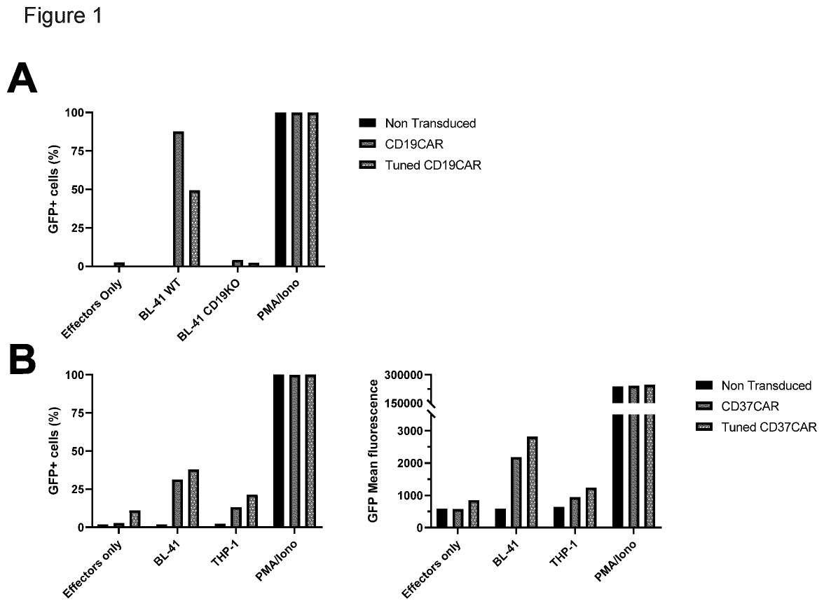

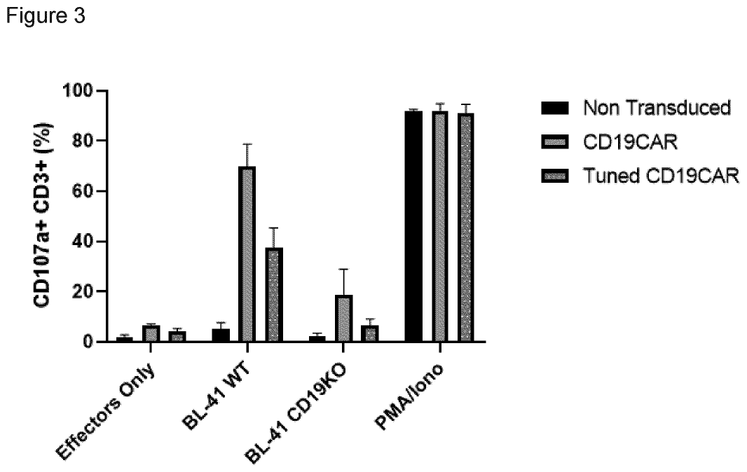

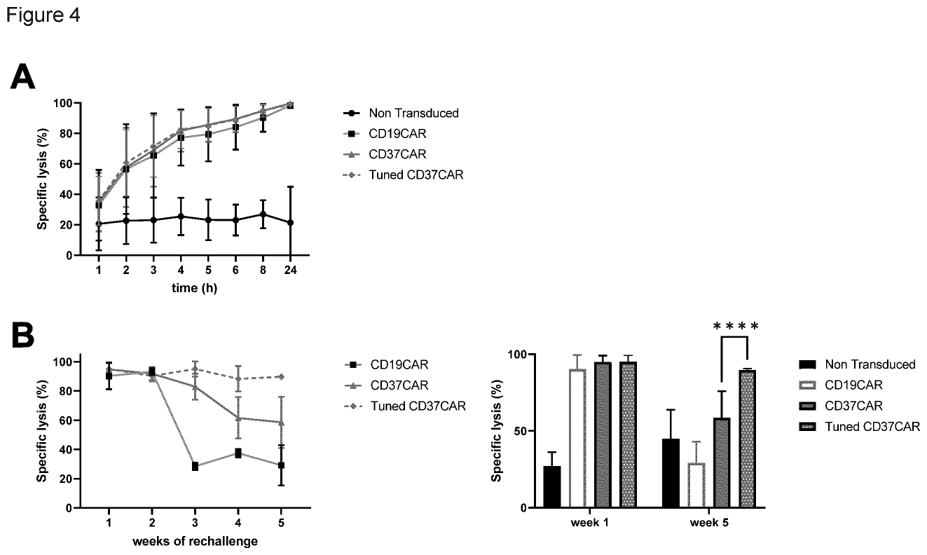

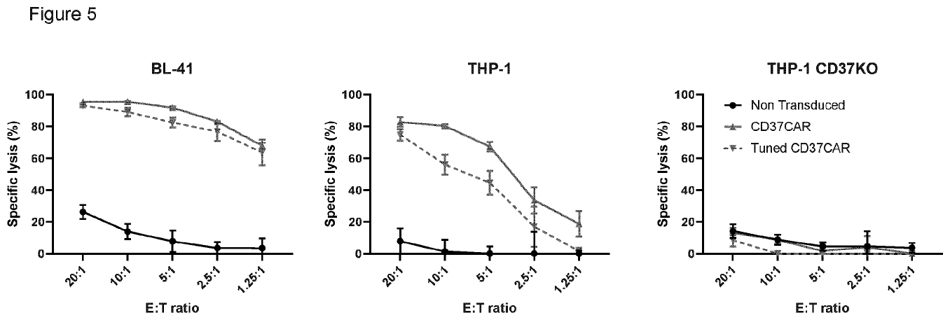

A polycistronic nucleic acid encoding a first chimeric protein comprising a scFv for specific binding to CD19 or CD37, a CD8α-hinge, a CD8α transmembrane domain and a coiled coil dimerization domain followed by a ribosomal skipping sequence (P2A here) before the second chimeric protein comprising a myristolated lipid-anchoring domain, a coiled coil dimerization domain, a 4-1BB costimulatory domain and a CD3ζ signaling domain was transduced into the T cell line J76, stably transduced to express GFP protein downstream of a NFAT response element (J76 NFAT^{GFP} reporter) (FIG. 1). These transduced or non-transduced J76 NFAT^{GFP} cells were co-incubated with target cells. For CD19CAR, the target cells were CD19+ and CD37^{high} target cells (BL-41), and CD19 knock out (CD19⁻) BL-41 line as a negative control. For CD37CAR constructs, CD37^{high} target cells (BL-41) and CD37^{low} target cells (THP-1) were used. In all conditions, mock transfected J76 NFAT^{GFP} cells were used. As shown, stimulation (i.e. GFP signal increase) only occurs in the presence of the targeted antigen. Similarly, primary T cells isolated from healthy donors' PBMCs (n=2) transduced or not with CAR constructs directed against CD19 (FIGS. 3 and 4) or CD37 (FIGS. 4 and 5) demonstrated specific activation and cytotoxicity against target cells expressing the corresponding antigen. These experiments demonstrate the ability of the Tuned CAR-system to elicit antigen-dependent T cells activation and cytotoxicity while being as efficient as prior art technologies such as second generation 4-1BB-CD3ζ CAR, i.e. CAR constructs comprising a costimulatory signaling domain for example a CD2ζ or 4-BB costimulatory signaling domain.

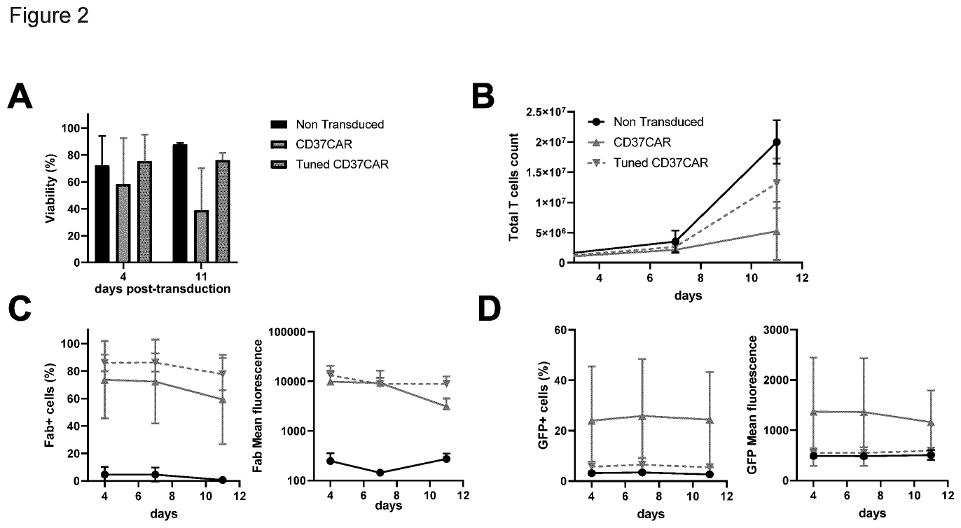

CAR T cells have been shown to be delayed in their expansion capacity, this is suspected to be due to the presence of the CAR construct which might signal without specific stimulation (tonic signaling). This tonic signaling, if too important as observed with CD37CAR, might even become toxic. We observed that J76 NFAT^{GFP} cells transduced with CD37CAR demonstrated a lower expansion capacity and viability than those expressing the Tuned CD37CAR (FIG. 2). Moreover, both CD37CAR and Tuned CD37CAR were expressed at similar levels as determined by staining with an anti-murine fragment antigen binding (anti-mFab) in flow cytometry (FIG. 2C) and Tuned CD37CAR demonstrated less basal T cell activation than CD37CAR (FIG. 2D).

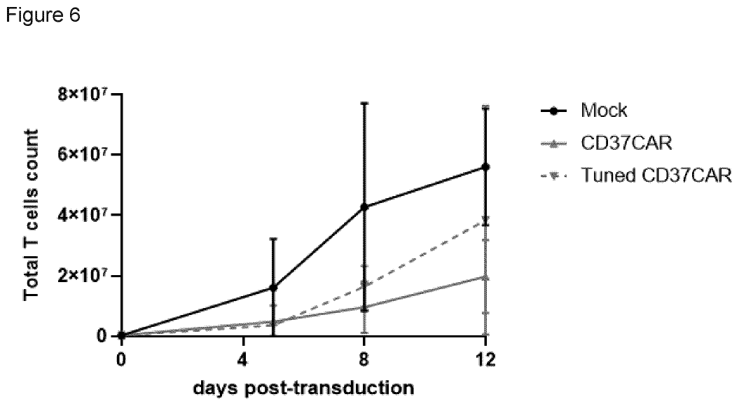

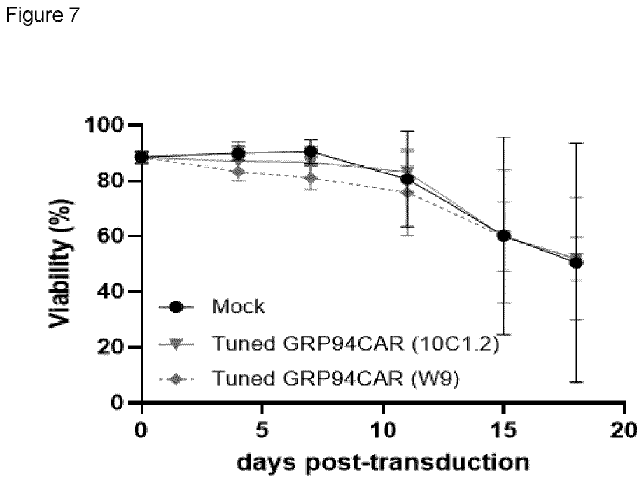

We confirmed these observations using PBMCs from healthy donors (n=2) transduced or not with either CAR, Tuned CD19CAR, Tuned CD37CAR or Tuned GRP94CAR (FIG. 6, 7, 8). Tuned CD37CAR PBMCs demonstrated a better expansion compared to CD37CAR (FIG. 6). GRP94 is a chaperone of the ER which can be expressed at the plasma membrane in cancer conditions (REF: PMID: 33802964), we have previously observed that primary T cells transduced with a second generation GRP94CAR were unable to grow and died within a few days. The same was true for J76 cell line, however the NK-92 line could tolerate its presence but a with some toxicity. However, in a Tuned GRP94CAR primary T cells, we observed that the cells were tolerating the construct (FIG. 7) and CAR positive cells were detected by flow (FIG. 7). Lastly, a second generation CD19CAR construct, described in the literature as being tonic, and a Tuned CD19CAR construct transduced into healthy donor PBMCs were used to assess the metabolic state at steady state of the T cells by using the Seahorse XF instrument with the Mito Stress Test Kit (FIG. 8). Tuned CD19CAR had a reduced OCR and

ECAR compared to CD19CAR (FIG. 8), suggesting that the Tuned CAR-system does not induce increase in metabolic activity during steady state.

Using the two cell models, i.e. J76 NFAT^{GFP} cells and primary T cells (PBMCs from healthy donors) we observed that the Tuned CAR-system was able to activate and induce cytotoxicity by T cells in an antigen-dependent manner (FIG. 1, 3, 4A, 5), reduce tonic signalling and/or toxicity (FIG. 2, 6, 7, 8) and improve persistence of T cells after multiple challenges (FIG. 4B)

1. A cell expressing a first and a second chimeric protein in the cell membrane;
wherein the first chimeric protein comprises, from N-terminal to C-terminal, an antigen binding unit, a transmembrane domain, and a first dimerization domain, but no functional CD3ζ signaling domain, and
wherein the second chimeric protein comprises, from N-terminal to C-terminal, a lipid-anchoring domain, a second dimerization domain, and a signaling domain.

2. The cell according to claim 1, wherein the antigen binding unit is an scFv.

3. The cell according to claim 1, wherein the cell is a T cell, an NK cell, or a macrophage.

4. The cell according to claim 1, wherein the signaling domain in the second chimeric protein comprises a CD3ζ signaling domain.

5. The cell according to claim 1, wherein the signaling domain in the second chimeric protein comprises a costimulatory domain and a CD3ζ signaling domain.

6. The cell according to claim 1, wherein the signaling domain in the second chimeric protein comprises a 4-1BB costimulatory domain and a CD3ζ signaling domain.

7. The cell according to claim 1, wherein the first chimeric protein comprises a hinge domain between the antigen binding unit and the transmembrane domain.

8. The cell according to claim 1, wherein the first dimerization domain specifically binds to the second dimerization domain with an affinity to convey a signal upon binding of target epitope.

9. The cell according to claim 1, wherein the first dimerization domain has a net positive charge and the second dimerization domain has a net negative charge.

10. The cell according to claim 1, wherein the first dimerization domain has a net negative charge and the second dimerization domain has a net positive charge.

11. The cell according to claim 1, wherein the first dimerization domain is represented by SEQ ID NO 2, 4, 6, 8, 10, 12, 14 or 16.

12. The cell according to claim 1, wherein the second dimerization domain is represented by SEQ ID NO 1, 3, 5, 7, 9, 11, 13 or 15.

13. The cell according to claim 1, wherein the first chimeric protein comprises a costimulatory domain.

14. The cell according to claim 1, wherein the second chimeric protein comprises two costimulatory domains.

15. The cell according to claim 1, wherein the antigen binding unit has a specific affinity for internal epitopes in the intracellular compartments which causes expression problems of conventional CAR constructs.

16. A nucleic acid encoding the first and/or second chimeric protein as defined in claim 1.

17. A pharmaceutical composition comprising the cell as defined in claim 1.

18. A method of treating cancer comprising the step of administering the pharmaceutical composition according to claim 17 to a patient in need thereof, and wherein the antigen binding unit specifically binds to a surface antigen on cancer cells under physiological conditions.

19. A method for bypassing expression problems of CARs by transducing a cell with the nucleic acid as defined in claim 16.

20. The cell according to claim 1, wherein the antigen binding unit has an unspecific affinity for internal epitopes in the intracellular compartments which causes expression problems of conventional CAR constructs.
